(19) United States
(12) Patent Application Publication
(10) Pub. No.: US 2024/0296564 A1
(43) Pub. Date: Sep. 5, 2024

(54) SYSTEM, APPARATUS, AND METHOD FOR AUTOMATED MEDICAL LASER OPERATION

(71) Applicants: Hanjin CHO, Vienna, VA (US); Chloe Selyn CHO, Vienna, VA (US)

(72) Inventors: Hanjin CHO, Vienna, VA (US); Chloe Selyn CHO, Vienna, VA (US)

(21) Appl. No.: 18/594,644

(22) Filed: Mar. 4, 2024

Related U.S. Application Data
(60) Provisional application No. 63/487,953, filed on Mar. 2, 2023.

(51) Int. Cl. G06T 7/00; G06T 17/00; G16H 30/40; G16H 50/70

(52) U.S. Cl. CPC G06T 7/0014; G06T 17/00; G16H 30/40; G16H 50/70; G06T 2207/20081

(57) ABSTRACT

A system, method, and apparatus for autonomous laser operation. The method includes performing an imaging scan of a treatment area, generating a scanned model of the treatment area based on the imaging scan, comparing the scanned model to a desired outcome model of the treatment area, generating laser operation instructions based on a difference between the scanned model and the desired outcome model, and operating a laser on the treatment area according to the laser operation instructions. The desired outcome model is generated based on the imaging scan and on prior data pertaining to the treatment area.

### CROSS-REFERENCE TO RELATED APPLICATIONS

This application claims priority to U.S. Provisional Application No. 63/487,953, filed Mar. 2, 2023, the entire contents of which are hereby incorporated by reference.

### BACKGROUND

Surgical lasers are particularly useful for a myriad of operations and procedures. For example, many dental procedures and operations may utilize a surgical laser for intraoral operations such as for root canals or to mold a tooth for a crown. The operation site in most cases needs to be measured and modeled carefully using an intraoral scanner so that the operation can be designed. For example, in a prosthodontic procedure a tooth or gum may need to be measured and then selectively shaped using a surgical laser which to create a surface which the crown, denture, or bridge, for example, then grasps. In other procedures, surface contours of the areas where missing teeth are to be replaced may need to be reproduced accurately so that the resulting prosthetic fits over the edentulous region with even pressure on the soft tissue.

Laser cutting systems have been implemented in various industries. For example, lasers have been used in computer-aided manufacturing systems to cut and produce various types of consumer goods, from automotive body panels to personalized jewelry. Typical systems rely on computer-aided design (CAD) figures in order to produce precise and replicable results. Surgical lasers, on the other hand, are controlled by the surgeon and rely on the surgeon's placement of the laser as opposed to being controlled by a computer-articulated robotic member. Further, unlike handheld drills or burrs, a handheld laser does not provide tactile feedback to the user and may be therefore harder to control when cutting. As a result, there is a need in the field to provide handheld or intraoral laser systems with the accuracy and precision of computer-aided laser systems.

### SUMMARY

According to at least one exemplary embodiment, a system, method, and apparatus for autonomous laser operation is disclosed. The method may include performing an imaging scan of a treatment area, generating a scanned model of the treatment area based on the imaging scan, comparing the scanned model to a desired outcome model of the treatment area, generating laser operation instructions based on a difference between the scanned model and the desired outcome model, and operating a laser on the treatment area according to the laser operation instructions. The desired outcome model may be generated based on the imaging scan and on prior data pertaining to the treatment area. The steps of the method may be repeated until the scanned model sufficiently resembles the desired outcome model, and may be repeated in real time. The desired outcome model may be generated by machine learning or artificial intelligence procedures that are trained on prior data, which may include one or more of training data sets and historical data of prior procedures relevant to the treatment area. The imaging scan may include three-dimensional volumetric data and may further include two-dimensional data.

Operating the laser may further include determining a situation of a source of the laser with respect to the treatment area, and controlling operation of the laser at one or more locations of the treatment area according to the laser operation instructions while accounting for the situation of the source of the laser. The situation of the source of the laser may include one or more of a position of the source of the laser, an orientation of the source of the laser, and a movement of the source of the laser. Controlling operation of the laser may further include one or more of activating the laser, deactivating the laser, directing the laser, and varying an intensity of the laser.

The system may include a laser module including a surgical laser and a guidance system, a scanning module including one or more of a 3D imaging device and a 2D imaging device, and a controller. The controller may be adapted to execute the steps of the method. The laser module and the scanning module may be disposed in a housing. The controller may further be adapted to determine a situation of the housing with respect to the treatment area, and control operation of the laser at one or more locations of the treatment area according to the laser operation instructions while accounting for the situation of the housing. The situation of the housing may include one or more of a position of the housing, an orientation of the housing, and a movement of the housing. The controller may further be adapted to control the operation of the laser by one or more of activating the laser, deactivating the laser, directing the laser, and varying an intensity of the laser.

An exemplary machine learning algorithm may be trained for various types of operations. For example, an embodiment may be trained to identify tooth decay to be removed, and then may direct the laser to remove the detected or identified decayed portions of the tooth. An exemplary embodiment may be used on both soft and hard tissue, or on any other contemplated material. Alternatively, other embodiments may be trained for other intra-or extra-oral operations, for example, hair, tattoos, skin lesions, or other abnormalities can be identified for removal by an exemplary embodiment.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1A shows an exemplary embodiment of an apparatus and system for automated medical laser operation.

FIG. 1B shows another exemplary embodiment of an apparatus and system for automated medical laser operation.

FIG. 2 shows an exemplary embodiment of a method for automated medical laser operation.

FIG. 3 shows another exemplary embodiment of a method for automated medical laser operation.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein are described in terms of sequences of actions to be performed by, for example, elements of a computing device. It should be recognized by those skilled in the art that the various sequences of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the at least one processor to perform the functionality described herein. Furthermore, the sequence of actions described herein can be embodied in a combination of hardware and software. Thus, the various aspects of the present invention may be embodied in a number of different forms, all of which have been contemplated to be within the scope of the claimed subject matter. In addition, for each of the embodiments described herein, the corresponding form of any such embodiment may be described herein as, for example, “a computer configured to” perform the described action.

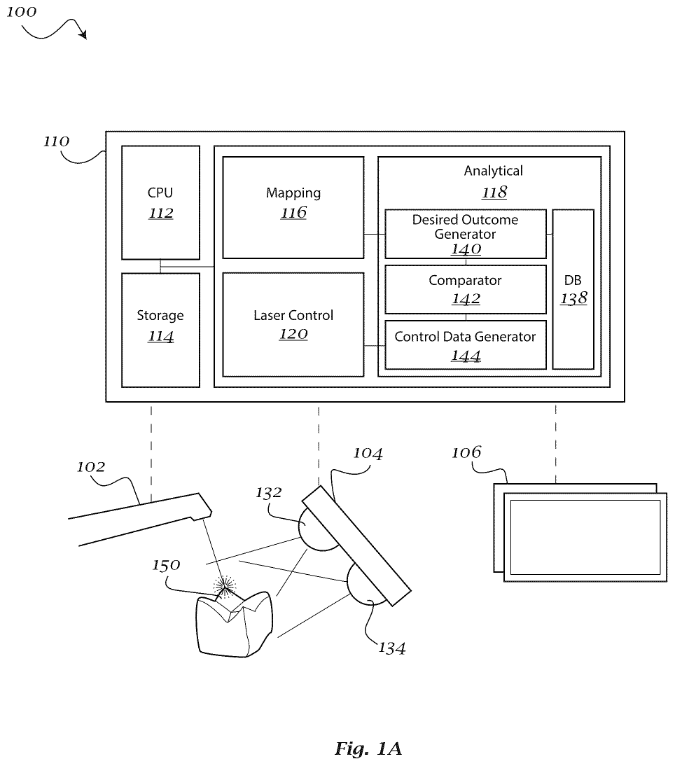

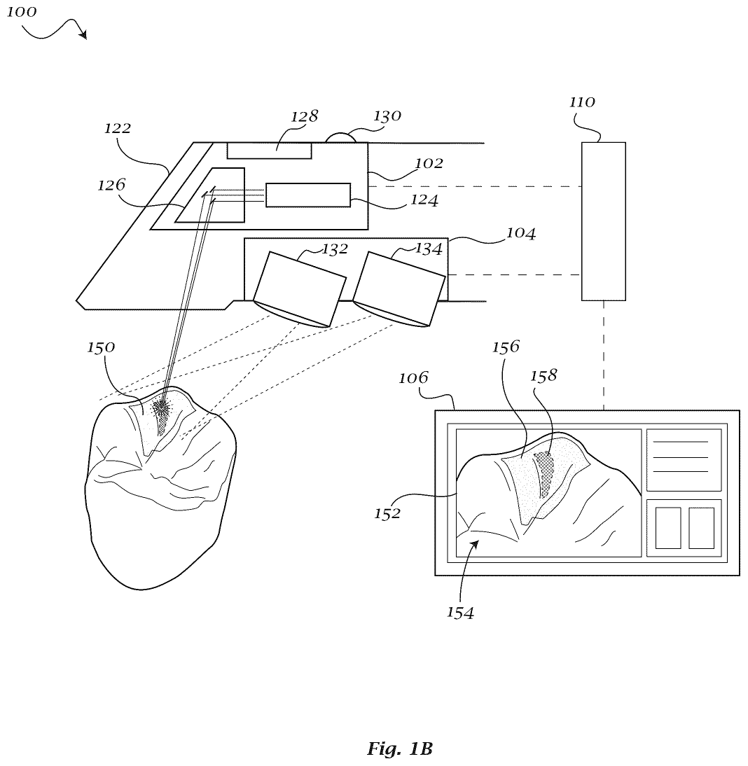

Referring to FIGS. 1A-1B, and according to at least one exemplary embodiment, a system for automated medical laser operation 100 is disclosed. System 100 may include a laser module 102, a scanning module 104, one or more displays 106, and a controller 110 communicatively coupled to the laser module, the scanning module, and the one or more displays. Controller 110 may include a processor 112, a non-volatile storage medium 114, communications buses for communicating with the various hardware components of system 100, networking components, and may generally be a computing device having components adapted to cause system 100 to function as described herein. Controller 110 may further include software that may be stored on the medium 114 and executable by processor 112. The software of controller 110 may include a mapping module 116, an analytical module 118, and a laser control module 120.

Laser module 102 may be disposed within a housing 122, and may include a surgical laser 124, a guidance system 126, a haptic system 128, and user inputs 130. Housing 122 may be sized and shaped as a handheld housing that can allow for easy manipulation of the housing by a user, so as to direct the laser towards desired regions in a treatment area 150 as necessary. Surgical laser 124 may be adapted for both invasive and non-invasive treatment of hard and soft tissue, and may be any surgical laser that enables system 100 to function as described herein.

Guidance system 126 may be adapted to activate, direct, and deactivate laser 124. Guidance system 126 may direct laser 124 with respect to housing 122. To that end, guidance system 126 may include elements such as motors, mirrors, movable or rotatable mirrors, arrays of digital micromirrors, and so forth, disposed within housing 122, and adapted to direct the beam or beams of laser 124 with respect to housing 122. For example, as housing 122 is moved with respect to the treatment area 150 by the user, guidance system 126 may be utilized to further direct the laser with respect to the housing as determined by controller 110, as described further below. In some exemplary embodiments, the laser may be guided using motorized controls that adjust the laser or using mirrors or other surfaces to guide or reflect the laser towards the desired location. In yet other exemplary embodiments, a motorized reflection device inside housing 122 may be provided, and/or a digital micromirror device which can control individual beams when an array of beams is used.

Haptic system 128 may provide haptic or tactile feedback to the user during use of laser module 102. For example, haptic feedback may be provided so as to mimic tactile feedback experienced by a surgeon when using a surgical tool that contacts hard or soft tissue. In some embodiments, the intensity of the haptic feedback further may vary depending on laser output. For example, a higher base level of haptic feedback may be provided for full-strength laser output and/or fast or bulk cutting and a lower base level of haptic feedback may be provided during minimum laser output and/or slow or precision cutting.

User input 130 may provide for user control of laser module 102. The user input may allow the user to control an on/off state of the laser 124, as well as an intensity of laser 124. To that end, user input 130 may be a rheostat-type input, may be pressure-sensitive, or may otherwise allow for variable or gradual input. For example, a pressure-sensitive or spring-loaded rotary finger switch may be provided on housing 122, or a pressure-sensitive foot pedal may be provided separately from the housing. Depressing the main user input may initiate operation of laser 124, and the output power and/or firing rate of the laser may be dependent on the level of input applied to the main user input. The intensity of laser 124 in response to the user input 130 may also be modulated as determined by controller 110, as described further below.

Scanning module 104 may be provided within housing 122, or may be provided separate therefrom. Scanning module 104 can include one or more 2D imaging devices 132, and one or more 3D imaging devices 134. The 2D imaging device 132 may be adapted to generate a two-dimensional image of the treatment area 150, and may be an optical camera, or any other imaging device that enables system 100 to function as described herein. The 3D imaging device 134 may be adapted to generate a three-dimensional scan of the treatment area 150, and may utilize LIDAR sensors, or any other three-dimensional sensor that enables system 100 to function as described herein. Imaging devices 132, 134 may communicate the acquired data to controller 110. Furthermore, in some exemplary embodiments, the scanning module 104 or 3D imaging device 134 may be adapted to track the positions of housing 122 and/or laser 124 in relation to the treatment area. In yet other exemplary embodiments, additional sensors or sensing methodologies may be provided to determine the positions of housing 122 and/or laser 124 in relation to the treatment area.

Displays 106 may display interfaces related to the functionality of system 100, as well as image data related to the functionality of system 100. Before or during an operation, displays 106 may display interfaces adapted to configure settings, parameters, and other relevant information based on the type of operation that is to be performed. For example, in embodiments directed towards dental surgery, such interfaces may include a menu listing including the various categories of dental surgery to be performed, such as hard tissue operations, soft tissue operations, and non-surgical operations. Further options may be provided for each category, for example, cavity preparation, crown, veneer, onlay, inlay preparation, alveoloplasty, and so forth, for hard tissue operations; gingivectomy, abnormal lesion excision or incision, and so forth, for soft tissue operations; and gingiva or skin laser massage, and so forth, for non-surgical operations. Further exemplary embodiments can provide interfaces for presetting and adjusting angle, timing, strength, and other parameters of the surgical laser for the selected procedure.

During surgery, displays 106 may display live imagery 152 of the treatment area 150. The imagery can include a real-time optical view of the treatment area or a portion thereof, which may be sourced, for example, from 2D imaging device 132, or any other camera device viewing the treatment area. The imagery can further include a real-time three-dimensional representation 154 of the treatment area or a portion thereof, which may be sourced from 3D imaging device 134 and processed via controller 110. Overlays may further be provided on, or in addition to, the three-dimensional representation of the treatment area. For example, an overlay may show a volumetric model 156 representing an area or volume to be surgically or non-surgically treated by laser 124. The overlay may be modified in real time as the treatment area is treated with a laser; for example, portions of the volumetric model overlay may be removed from the display as the corresponding treatment area is removed by the laser. As another example, an overlay may show an additional area, which may be a two-or three-dimensional area, for example as an array of dots or a translucent region, which shows the area 158 that is to be imminently treated by laser 124. The overlay may be modified and repositioned in real time as housing 122 is moved in the treatment area and as the treatment area 150 is treated by laser 124. The overlays may be provided by controller 110.

Controller 110 may include a mapping module 116, an analytical module 118, and a laser control module 120. Mapping module 116 may receive real-time 2D data and 3D data of the treatment area from imaging devices 132, 134, respectively. Mapping module 116 may utilize the received 2D and 3D data to generate real-time models of the treatment area 150. For example, two-dimensional boundaries of the treatment area 150 may be constructed, and/or 3D volumetric models, including hard and soft tissue, of the treatment area may be constructed. Accordingly, a real-time combined 2D/3D model of the treatment area may be generated. The real-time combined 2D/3D model of the treatment area may be generated and may further be output to machine learning module 118.

The analytical module 118 may include a database 138, a desired outcome generator 140, a comparator 142, and a control data generator 144. In some embodiments, database 138 may contain a training dataset derived from training data and/or historical data of prior surgical operations. For example, a standardized training dataset containing pre-and post-treatment three-dimensional volumetric data pertaining to various surgical procedures may be provided in database 138. Additionally or alternatively, an accumulated training dataset having pre-and post-treatment three-dimensional volumetric data from past, performed surgical procedures may be provided in database 138. The analytical module 118 may utilize machine learning techniques, and/or artificial intelligence techniques, so as to be trained on the training datasets for various treatment areas so as to predict and generate a three-dimensional volumetric shape of the various treatment areas as they would appear post-surgery. Additionally, in exemplary embodiments, image classification may be used to identify abnormalities based on the 2D and/or 3D scans of the treatment area. The analytical module 118 may accordingly be trained to identify and appropriately treat any identified abnormalities, if necessary.

The desired outcome generator 140 of the analytical module 118 may receive the real-time combined 2D/3D model of the treatment area, and, based on the received real-time model, combined with the training received from the training datasets, generate a desired outcome 3D model of the treatment area. In other words, the desired outcome 3D model may be based on real-time 2D/3D data of the treatment area presently under surgical operation, modified by the trained machine learning module, resulting in a generated ideal or desired outcome volumetric 3D model of the treatment area post-surgery.

The comparator 142 may receive the desired 3D model as well as the real-time combined 2D/3D model of the treatment area and compare the desired model to the real-time combined model. Based on this comparison, the comparator 142 can determine parameters for the surgical laser operation to be performed so that the treatment area resulting from the surgical operation will be the same or substantially similar as the treatment area according to the desired 3D model. The comparator can determine various parameters for the surgical operation, such as the location in the treatment area where the operation is to be performed, the extent to which the operation should be performed, the tissue that should be treated by the laser, and the number of steps required for the complete operation. A surgical operation may be broken down into a plurality of steps, wherein each step may perform a partial treatment of the treatment area. After each step, the treatment area may be rescanned by the 2D and 3D imaging devices 132, 134, and the resulting data input into comparator 142, so as to determine the parameters of the next step of the surgical operation.

The control data generator 144 may receive the topological data from comparator 142 and output instructions and parameters to laser control module 120 for the operation of laser 124 that are necessary to complete the next subsequent surgical operation. Laser control module 120, in turn, may further determine the real-time position, orientation, and operating status of laser 124, and may control the operation of the laser based on both the instructions and parameters received from control data generator 144, as well as the real-time position, orientation, and operating status of the laser. For example, as the user moves housing 122 over the treatment area, laser control module 120 may utilize inputs from imaging devices 132, 134, from sensors determining the position and orientation of the laser, as well as any other necessary inputs, to control the operation of the laser. Laser control module 120 may control operation of the laser by controlling laser guidance system 126 to direct the beam of laser 124 to the appropriate location in the treatment area, to vary the intensity of laser 124, and to activate and deactivate laser 124 as necessary. Therefore, even as housing 122 is moved by the user, more precise movement of laser 124 may be performed by laser control module 120; similarly, if the housing 122 is held still or substantially still by the user, precise movement of laser 124 may nevertheless be performed by laser control module 120. Additionally, in response to sudden or unexpected movements of housing 122, or removal of the housing from the treatment area, laser 124 may be deactivated by laser control module 120. The laser may then be reactivated when housing 122 is returned to the appropriate location in the treatment area. In regular operation, however, laser control module 120 may control laser 124 according to the parameters received from control data generator 144, such that the surgical operation can be performed according to the topological data generated by comparator 142. For example, the laser control data that is based on topological data may indicate the portions of the treatment area to which laser 124 is to be applied, as well as portions to which the laser should not be applied. Accordingly, laser control module 120 may deactivate the laser 124 in real time as the user directs housing 122 over portions that should not be operated on, while activating laser 124 when the housing is proximate the portions to which the laser is to be applied. Further, exemplars embodiment may control the magnitude or power of laser 124 based on the distance of housing 122 from the appropriate location in the treatment area.

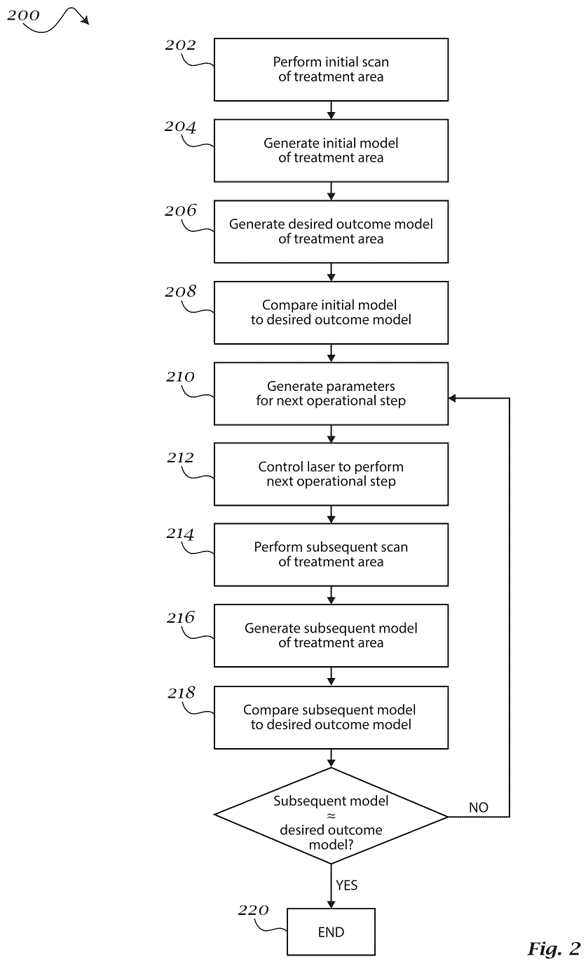

Turning to FIG. 2, an exemplary method for automated laser operation 200 is disclosed. At step 202, a treatment area may be selected and an initial 2D and 3D scan of the treatment area may be performed. The 2D and 3D data may then be input into analytical module 118. At step 204, mapping module 116 may analyze the received 2D and 3D data and generate an initial model of the scanned treatment area. Subsequently, the generated initial model may be input into analytical module 118.

At step 206, the analytical module may generate a desired outcome 3D model of the treatment area, based on the initial model of the treatment area. In some embodiments, the analytical module may also utilize machine learning algorithms and artificial intelligence techniques to generate the desired outcome model of the treatment area. To that end, the analytical module may be trained on training datasets containing historical data such as pre-and post-treatment three-dimensional volumetric data pertaining to prior relevant surgical procedures.

At step 208, the analytical module may compare the initial model of the scanned treatment area to the desired outcome model of the treatment area, and, at step 210, generate parameters for a next operational step of a laser operation based on the comparison. The analytical module may determine parameters for the next operational step such that, subsequent to the next operational step, the treatment area more resembles the desired outcome model. Such parameters may include topological data indicating the location of the treatment area where the next operational step is to be performed as well as operational parameters of the laser, pertaining to the next operational step. The analytical module may then generate laser control data based on the determined parameters, and output the laser control data to laser control module 120. At step 212, laser 124 may be controlled according to the laser control data to carry out the next operational step.

After the laser operation of step 212, a further 2D and 3D scan of the treatment area may be performed and a subsequent model of the scanned treatment area may be generated, at step 214. At step 216, the subsequent model may be compared to the desired outcome model by the analytical module. At step 218, the analytical module may determine whether the subsequent model sufficiently resembles the desired outcome model, and, if so, the operation may terminate at step 220. However, if the subsequent model does not sufficiently resemble the desired outcome model, the method may return to step 210, wherein parameters for the next operational step may be generated and laser control data output to the laser control module. Laser 124 may be controlled according to the newly-output laser control data to carry out the next step of the operation according to the latest generated parameters. Steps 210-218 may then be repeated until the operation is determined to be complete according to comparison of the scanned treatment area and the desired outcome model.

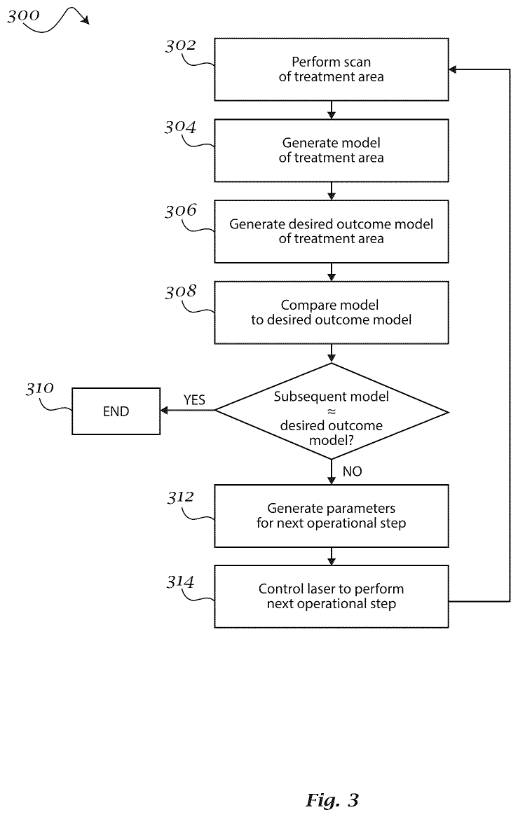

Turning to FIG. 3, another exemplary method for automated laser operation 300 is disclosed. At step 302, a treatment area may be selected and a 2D and 3D scan of the treatment area may be performed. The 2D and 3D data may then be input into analytical module 118. At step 304, mapping module 116 may analyze the received 2D and 3D data and generate a model of the scanned treatment area. Subsequently, the generated model may be input into analytical module 118.

At step 306, the analytical module may generate a desired outcome 3D model of the treatment area, based on the model of the scanned treatment area. In some embodiments, the analytical module may also utilize machine learning algorithms and artificial intelligence techniques to generate the desired outcome model of the treatment area. To that end, the analytical module may be trained on training datasets containing historical data such as pre-and post-treatment three-dimensional volumetric data pertaining to prior relevant surgical procedures.

At step 308, the analytical module may compare the model of the scanned treatment area to the desired outcome model of the treatment area. The analytical module may then determine whether the scanned model sufficiently resembles the desired outcome model, and, if so, the operation may terminate at step 310. However, if the scanned model does not sufficiently resemble the desired outcome model, the method may proceed to step 312, wherein the analytical module may generate parameters for a next operational step of a laser operation based on the comparison. The analytical module may determine parameters for the next operational step such that, subsequent to the next operational step, the treatment area more resembles the desired outcome model. Such parameters may include topological data indicating the location of the treatment area where the next operational step is to be performed as well as operational parameters of the laser, pertaining to the next operational step. The analytical module may then generate laser control data based on the determined parameters, and output the laser control data to laser control module 120. At step 314, laser 124 may be controlled according to the laser control data to carry out the next operational step. The process may then return to step 302 and may be repeated until the operation is determined to be complete according to comparison of the scanned treatment area and the desired outcome model.

In the exemplary embodiment of method 300, the desired outcome model may be updated, revised, or regenerated after every operational step. For example, minor adjustment or modification of the desired outcome model may be advantageous after each operational step due to a changing of the surface of the treatment area, such as previously concealed portions of the surface being revealed as a consequence of the operational step. As a further example, if, in a dental operation, two teeth in close proximity and in tight contact with each other, an exact shape of the contact area or interface between the teeth may not be known due to being hidden by adjacent teeth. Consequently, such areas may not be part of an initial scan of the treatment area. Therefore, such portions of the desired outcome model may initially be guessed or approximated by the desired outcome generator from the initial scan data, but may need to be adjusted and updated further as the area is revealed and re-scanned after each operational step.

It should be appreciated that, in the exemplary methods, each operational step may have a duration time t, wherein t may vary from the scale of seconds to the scale of fractions of a second, milliseconds, microseconds, nanoseconds, and so forth. Accordingly, the parameters for each operational step may be generated and provided for the appropriate time scale. In other words, in some embodiments, the parameters for an operational step may be generated so as to provide for several seconds, or several fractions of a second, of laser operation prior to the subsequent scan, comparison, and generation of subsequent parameters. In other embodiments, the parameters for an operational step may be generated so as to provide for laser operation for a duration on the scale of microseconds, milliseconds, or nanoseconds prior to the subsequent scan, comparison, and generation of subsequent parameters, effectively resulting in a real-time tracking of the laser operation, wherein scans, comparisons, parameter generation and laser control are being performed continuously.

In some exemplary embodiments, manual operation capability may also be provided. If desired by the user, the automated laser operation process may be temporarily interrupted, so that the user can manually perform operations as generally known in the art. To that end, an indicator, for example a non-surgical laser pointer may be provided, so as to indicate the location where the manual operations are to be performed. Subsequent to the manual operations, the automated laser operation process may be recommenced, starting with a scan of the treatment area according to step 214 or the like.

Further exemplary embodiments may include additional features complementing the automated laser operation apparatus. For example, an irrigation system may be provided to cool down the treatment area during the laser treatment, so as to reduce the total laser-induced thermal damage area and prevent tissue carbonization. Some exemplary embodiments may provide for connection to a liquid reservoir containing, for example, water, saline, or any other contemplated liquid for applying to the treatment area before, during, or after an operation. Irrigation devices may be manually operated by the user or may be operated by controller 110 according to parameters generated for a particular operation. autonomously operated by the processor and memory. In some embodiments, analytical module 118 may include provisions for irrigation and may be trained to determine when and under which conditions irrigation may be beneficial or necessary and provide parameters for operation of irrigational equipment accordingly. Additionally, in some embodiments, temperature measuring devices may be provided at the treatment area and communicatively coupled to controller 110 so as to provide irrigation as necessary based on temperature thresholds, which may be determined by the trained analytical module 118.

In further exemplary embodiments, controller 110 may provide feedback or instructions to the user, for example via haptic system 128 or interfaces on displays 106. For example, as housing 122 is tracked proximate the treatment area, the controller may indicate to the user to move the housing closer in proximity to desired portions of the treatment area, for example, portions where further operations need to be carried out.

In further exemplary embodiments, image classification may be used to identify abnormalities based on the 2D and/or 3D scans of the treatment area. Analytical module 118 may accordingly be trained to identify and appropriately treat any identified abnormalities, if necessary. Controller 110 may indicate the presence of abnormalities to the user and direct the user to move housing 122 proximate the abnormality for further treatment.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for autonomous medical laser operation, comprising:
a) performing an imaging scan of a treatment area;
b) generating a scanned model of the treatment area based on the imaging scan;
c) comparing the scanned model to a desired outcome model of the treatment area;
d) generating laser operation instructions based on a difference between the scanned model and the desired outcome model; and
e) operating a laser on the treatment area according to the laser operation instructions;
wherein the desired outcome model is generated based on the imaging scan and on prior data pertaining to the treatment area.

2. The method of claim 1, further comprising repeating steps a-e until the scanned model sufficiently resembles the desired outcome model.

3. The method of claim 2, wherein steps a-e are continuously repeated in real time.

4. The method of claim 1, wherein the prior data pertaining to the treatment area includes one or more of training data sets and historical data of prior procedures relevant to the treatment area.

5. The method of claim 4, wherein the desired outcome model is generated by machine learning or artificial intelligence procedures that are trained on the prior data.

6. The method of claim 1, wherein the imaging scan comprises three-dimensional volumetric data.

7. The method of claim 6, wherein the imaging scan further comprises two-dimensional data.

8. The method of claim 1, wherein operating the laser further comprises:
determining a situation of a source of the laser with respect to the treatment area; and
controlling operation of the laser at one or more locations of the treatment area according to the laser operation instructions while accounting for the situation of the source of the laser;
wherein the situation of the source of the laser includes one or more of a position of the source of the laser, an orientation of the source of the laser, and a movement of the source of the laser; and
wherein controlling operation of the laser includes one or more of activating the laser, deactivating the laser, directing the laser, and varying an intensity of the laser.

9. A system for autonomous medical laser operation, comprising:
a laser module including a surgical laser and a guidance system;
a scanning module including one or more of a 3D imaging device and a 2D imaging device; and
a controller;
wherein the controller is adapted to:
a) receive imaging data of a treatment area from the scanning module;
b) generate a scanned model of the treatment area based on the imaging data;
c) compare the scanned model to a desired outcome model of the treatment area;
d) generate laser operation instructions based on a difference between the scanned model and the desired outcome model; and
e) operate the laser on the treatment area according to the laser operation instructions;
wherein controller is further adapted to generate the desired outcome model based on the imaging data and on prior data pertaining to the treatment area.

10. The system of claim 9, wherein the controller is further adapted to repeat steps a-e until the scanned model sufficiently resembles the desired outcome model.

11. The system of claim 10, wherein the controller is further adapted to repeat steps a-e continuously in real time.

12. The system of claim 9, wherein the prior data pertaining to the treatment area includes one or more of training data sets and historical data of prior procedures relevant to the treatment area.

13. The system of claim 12, wherein the controller is further adapted to generate the desired outcome model by machine learning or artificial intelligence procedures that are trained on the prior data.

14. The system of claim 9, further comprising a housing, wherein the laser module is disposed within the housing.

15. The system of claim 14, wherein the scanning module is disposed within the housing.

16. The system of claim 14, wherein the controller is further adapted to:
determine a situation of the housing with respect to the treatment area; and
control operation of the laser at one or more locations of the treatment area according to the laser operation instructions while accounting for the situation of the housing.

17. The system of claim 16, wherein the situation of the housing includes one or more of a position of the housing, an orientation of the housing, and a movement of the housing.

18. The system of claim 17, wherein the controller is adapted to control the operation of the laser by one or more of activating the laser, deactivating the laser, directing the laser, and varying an intensity of the laser.
